SEQUENCE LISTING (2 sequences)

SEQ ID NO: 1 - protein, 29 aa, unidentified
  REGION 1..29: note = Wild-type peptide
EQRASPLTSIISAVVGILLVVVLGVVFGI

SEQ ID NO: 2 - protein, 29 aa, unidentified
  REGION 1..29: note = mutated peptide
EQRASPLTSIISAVEGILLVVVLGVVFGI

(19) United States
(12) Patent Application Publication
(10) Pub. No.: US 2025/0082739 A1
(43) Pub. Date: Mar. 13, 2025

(54) METHODS FOR DESIGNING IMMUNOTHERAPIES AND TREATMENTS USING SAME

(71) Applicant: The Regents of the University of California, Oakland, CA (US)

(72) Inventors: Maurizio ZANETTI, La Jolla, CA (US); Hannah K. CARTER, San Diego, CA (US)

(21) Appl. No.: 18/621,627

(22) Filed: Mar. 29, 2024

Related U.S. Application Data
(60) Provisional application No. 63/455,746, filed on Mar. 30, 2023.

(51) Int. Cl. A61K 39/00; G01N 33/574

(52) U.S. Cl. CPC A61K 39/0011; G01N 33/5743; A61K 2039/5158; A61K 2039/54; A61K 2039/543; A61K 2039/55555; G01N 2333/70539

(57) ABSTRACT

Provided are methods for identifying and selecting an antigen that will be efficacious as an immune-stimulating antigen in vivo, comprising: (i) providing a plurality of potential neoantigens, wherein optionally the plurality of potential neoantigens comprise a plurality of peptides; (ii) identifying if there is a contextual-linked recognition of a candidate neoantigen by a CD4 and/or a CD8 T cell; (iii) identifying if a source protein of the candidate neoantigen, or the neoantigen, is present in a subcellular location source known to contain immunogenic peptides, and the neoantigen is enriched in at least one intracellular compartment or the neoantigen is depleted in at least one intracellular compartment; (iv) identifying if there is a redundancy of presentation of the neoantigen by at least two different major histocompatibility complex (MHC) proteins; and (v) if a potential neoantigen meets the criteria of: (ii) and (iii); (ii) and (iv); (iii) and (iv); or (ii), (iii) and (iv), the potential neoantigen is identified as an antigen that will be efficacious as an immune-stimulating antigen in vivo.

### RELATED APPLICATIONS

This U.S. utility patent application claims the benefit of priority under 35 U.S.C. § 119(e) to U.S. Provisional Patent Application Ser. No. (U.S. Ser. No.) 63/455,746, Mar. 30, 2023. The aforementioned application is expressly incorporated herein by reference in their entirety and for all purposes.

### STATEMENT AS TO FEDERALLY SPONSORED RESEARCH

This invention was made with government support under CA220009 awarded by the National Institutes of Health (NIH). The government has certain rights in the invention.

### REFERENCE TO ELECTRONIC SEQUENCE LISTING

The application contains a Sequence Listing which has been submitted electronically in.XML format and is hereby incorporated by reference in its entirety. Said.XML copy, created on Mar. 28, 2024, is named “0321.151880.xml” and is 3,446 bytes in size. The sequence listing contained in this.XML file is part of the specification and is hereby incorporated by reference herein in its entirety.

### TECHNICAL FIELD

This invention generally relates to immunology and immunotherapies. In alternative embodiments, provided are methods for identifying putative neoantigens based on these criteria: (i) potential for Linked recognition by CD4 and CD8 T cells; (ii) intracellular Location of the source protein; (iii) redundancy of neoantigen presentation by MHC alleles, and methods for using these neoantigens in disease therapies, for example, for identifying putative cancer neoantigens to be used in methods for treating cancer by personalized cancer vaccines or adoptive cell therapies.

### BACKGROUND

The ascent of cancer immunotherapy in the past decade equals decades of doubts on the role of immunity to fight cancer. Although success has been reported in numerous instances, confounding data demand we reassess how immunotherapy can leverage the cancer genome more efficiently. Neoantigens represent the most innovative aspect of the cancer immunotherapy revolution and the most direct link between cancer genome and immunity. Neoantigens form the basis of the original concept of immune surveillance where mutations introduce “foreignness” and T cells specific for mutant peptides are better at eluding thymic selection. Neoantigens are, therefore, the truest embodiment of Burnet's immune surveillance paradigm and the hallmark of personalized immunotherapy, but limitations to clinical translation are considerable.

Factors that influence the immunogenicity of neoantigens fall into three categories: (i) context, (ii) binding affinity for the Major Histocompatibility Complex (MHC) molecules, and (iii) the T cell repertoire of the individual. Context-dependent factors include the MHC and the fact that in cancer cells the MHC molecules can undergo loss of heterozygosity and accumulate mutations, hampering peptide presentation by the tumor cell. But possibly the more relevant context-dependent factor is the complex interplay between the MHC and driver mutations that emerge during tumor evolution.

From inception the adaptive T cell response is regulated by specialized cells: dendritic cells, macrophages and B cells (the professional antigen presenting cells, APC). An operational rule in T cell activation is linked (or associative) recognition of antigen. This posits that optimal T cell activation needs contextual help by another T cell (T-T cooperation), where both T cells respond to different antigenic peptides presented by the same APC. T-T cooperation exists between CD4 and CD8 T cells and also between two CD4 T cells. In both instances CD4 T cells serve the important role of facilitators. Thus, CD4 T cell help positively influences programming and post-programming of activated CD8 T cells, phases considered to be essential to the long term maintenance of memory CD8 T cells and their precursors. Likewise, T-T cooperation among two CD4 T cells is a functional switch to breach self-tolerance against tumor antigens leading to tumor control.

The identification and selection of mutant peptides with immunogenic characteristics (neoantigens) remains complex, hindering the clinical success of neoantigen (personalized) vaccines.

### SUMMARY

In alternative embodiments, provided are methods for identifying and selecting an antigen that will be efficacious as an immune-stimulating antigen in vivo,
wherein optionally the antigen that will be efficacious as an immune-stimulating antigen in vivo as (or in) a vaccine,
wherein optionally the antigen that will be efficacious as a T cell immune activating antigen,
the method comprising:
(i) providing a plurality of potential neoantigens, wherein optionally the plurality of potential neoantigens comprise a plurality of peptides, wherein optionally the peptides are between about 5 to 50 amino acids in length, or between about 2 to 25 amino acid in length;
(ii) identifying if there is a contextual-linked recognition of a candidate neoantigen (optionally a vaccine candidate neoantigen) by a CD4 and/or a CD8 T cell;
(iii) identifying if a source protein of the candidate neoantigen, or the neoantigen, is present in a subcellular location source known to contain immunogenic peptides, and
the neoantigen is enriched in at least one intracellular compartment or the neoantigen is depleted in at least one intracellular compartment,
and optionally the neoantigen is enriched when at least about 10%, 15%, 20% or 25% more amount of neoantigen is seen as compared to other neoantigens in the same cell compartment in the cell;
and optionally the neoantigen is depleted is when at least about 10%, 15%, 20% or 25% less amount of neoantigen is seen as compared to other neoantigens in the same cell compartment in the cell,
and optionally the cell compartment where the neoantigen is enriched is: cytoplasm, a NELF complex, nucleoplasm, a centrosome, a chromosome, a nucleolus, a nucleus or a combination thereof,
and optionally the cell compartment where the neoantigen is depleted is: Mitochondrial matrix and/or mitochondrion;
(iv) identifying if there is a redundancy of presentation of (or specific binding to) the neoantigen by at least two different major histocompatibility complex (MHC) proteins,
wherein optionally the neoantigen specifically binds a class 1 MHC polypeptide on a CD8+ cytotoxic T cell, and the neoantigen is a peptide 8 to 11 amino acids in length,
wherein optionally the neoantigen specifically binds a class II MHC polypeptide on a CD4+ helper T cell, and the neoantigen is a peptide 13 to 25 amino acids in length; and
(v) if a potential neoantigen meets the criteria of: (ii) and (iii); (ii) and (iv); (iii) and (iv); or (ii), (iii) and (iv), the potential neoantigen is identified as an antigen that will be efficacious as an immune-stimulating antigen in vivo,
wherein optionally the immune-stimulation comprises generating a T cell based immune response, and optionally the T cell based immune response comprises a CD4+ helper T cell or a CD8+ cytotoxic T cell stimulation directed to the neoantigen.

In alternative embodiments of methods as provided herein:
the neoantigen is a cancer or a tumor neoantigen;
the neoantigen is derived from an infectious agent optionally the infectious agent is a virus, a bacteria, a protozoan or a fungus;
the neoantigen is presented by (or is specifically bound by) a Class I MHC protein and a Class II MHC protein;
the major histocompatibility complex (MHC) protein is a Class I MHC protein or a Class II MHC protein,
the antigen and neoantigen is a tumor or cancer antigen and neoantigen;
the tumor or cancer is a melanoma, lung or bladder tumor or cancer; and/or
the antigen and/or neoantigen are derived from or comprise an epitope capable of generating an immune response, wherein the epitope is found in or on or derived from an infectious agent, optionally the infectious agent is a virus, a bacteria, a protozoan or a fungus.

In alternative embodiments, provided are methods for designing a vaccine against (directed to) a neoantigen comprising identifying and selecting a neoantigen using a method as provided herein.

In alternative embodiments, provided are methods for treating a tumor or a cancer comprising:
(a) exposing a T cell in vivo or ex vivo to a cancer or tumor neoantigen, wherein the cancer neoantigen is identified and selected using a method as provided herein;
wherein optionally the T cell is an autologous T cell, or the T cell is a CD4 or CD8 T cell; and
(b) (i) if the T cell is exposed ex vivo to the cancer or tumor neoantigen, administering the cancer or tumor neoantigen-exposed T cell to an individual in need thereof; or
(ii) if the T cell is exposed in vivo to the cancer or tumor neoantigen, a vaccine comprising the identified and selected cancer neoantigen is administered to the individual in need thereof.

In alternative embodiments, provided are methods for making a vaccine
comprising a neoantigen comprising:
(a) identifying and selecting at least one neoantigen, or antigen identified as efficacious as an immune-stimulating antigen in vivo as (or in) a vaccine, using the method as provided herein; and,
(b) formulating the at least one neoantigen or antigen with a vaccine formulation, optionally the vaccine formulation comprises a sterile saline.

In alternative embodiments, the at least one neoantigen, or antigen identified as efficacious as an immune-stimulating antigen in vivo, comprises at least one peptide.

In alternative embodiments, the at least one neoantigen, or antigen identified as efficacious as an immune-stimulating antigen, or the at least one peptide, is formulated in a liposome, a polymeric nanoparticle or a virus-like particles (VLP).

In alternative embodiments, a vaccine as provided herein or used in a method as provided herein further comprises an adjuvant or is administered to an individual in need thereof with an adjuvant, wherein optionally the adjuvant comprises an aluminum salt, MF59™ (or, squalene (4.3%) in citric acid buffer with stabilizing nonionic surfactants TWEEN 80™ (0.5%) and SPAN 85™ (0.5%)), and/or a CpG oligodeoxynucleotide.

In alternative embodiments, the at least one neoantigen, or antigen identified as efficacious as an immune-stimulating antigen in vivo, is loaded ex vivo onto a dendritic cell (DC), and the DC is thereupon administered to an individual in need thereof.

In alternative embodiments, provided are methods for treating a cancer or a tumor, or an infectious disease, comprising administering to an individual in need thereof a vaccine made by a method as provided herein, wherein optionally the vaccine is administered by a subcutaneous, intramuscular, or intranasal route.

In alternative embodiments, provide are methods for making a personalized adoptive cell therapy with an autologous T cell rendered neoantigen specific and expanded ex vivo comprising:
(a) identifying and selecting at least one neoantigen, or antigen identified as efficacious as an immune-stimulating antigen using a method as provided herein;
(b) stimulating ex vivo an autologous T cell from an individual in need thereof with at least one neoantigen, or antigen identified as efficacious as an immune-stimulating antigen; and
(c) administering to an individual in need thereof the ex vivo-stimulated autologous T cell.

In alternative embodiments, the ex vivo neoantigen-stimulated autologous T cell is administered directly into or approximate to (optionally within between about 1 to 50 mm to) a tumor site, or infected site, of an individual in need thereof.

In alternative embodiments, the ex vivo-stimulated autologous T cell is administered to the individual in need thereof with an immune checkpoint inhibitor, and optionally the immune checkpoint inhibitor comprises an anti-PD-1 or anti-CTLA-4 antibody.

In alternative embodiments, provided are methods treating a disease or condition comprising use of a personalized adoptive cell therapy with an autologous T cell rendered neoantigen-specific and expanded ex vivo comprising:
(a) identifying and selecting at least one neoantigen, or antigen identified as efficacious as an immune-stimulating antigen using a method as provided herein;
(b) stimulating ex vivo an autologous T cell from an individual in need thereof with at least one neoantigen, or antigen identified as efficacious as an immune-stimulating antigen; and
(c) administering to an individual in need thereof the ex vivo-stimulated autologous T cell.

In alternative embodiments provided are uses of an ex vivo-stimulated autologous T cell to treat a cancer or a tumor, wherein the autologous T cell is stimulated using the method as provided herein.

In alternative embodiments provided are uses of neoantigen to treat a cancer or a tumor, wherein the neoantigen is selected using a method as provided herein, wherein optionally the neoantigen is formulated in a vaccine and administered to an individual in need thereof.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1A-G illustrate an overview of location features from eluted peptides and T-cell assayed neoepitopes from Immune Epitope Database (IEDB):

FIG. 1A illustrates a scatterplot of clustered UMAP (Uniform Manifold Approximation and Projection (UMAP)) location features for source proteins of the Immune Epitope DataBase (IEDB) T-cell assayed neoepitopes with COSMIC (Catalogue Of Somatic Mutations In Cancer) (Wellcome Sanger Institute, Hinxton, Cambridgeshire, UK) cancer genes highlighted;

FIG. 1B illustrates a table annotating location clusters from the scatterplot with highlighted COSMIC cancer genes;

FIG. 1C-D graphically illustrate area under the receiver operating characteristic curve (AUROC), the true positive rate a function of false positive rate (FIG. 1C) and area under the precision-recall curve (AUPRC) (FIG. 1D) for 10-fold cross-validation using a Random Forest model to predict protein elution in 721.221 B cells incorporating location, matched expression, or both;

FIG. 1E-F graphically illustrate AUROC (FIG. 1E) and AUPRC (FIG. 1F) for 10-fold cross-validation using a RANDOM FOREST™ (Leo Breiman; Adele Cutler) model to predict immunogenicity in IEDB assayed neopeptides incorporating peptide affinity, stability, and foreignness with and without parent protein location features; and

FIG. 1G graphically illustrates a barplot of the exemplary model feature importance, including UMAP1+UMAP2; Affinity in nM; Stability (T-half (h)); and, Foreignness, as a function of feature importance,
as discussed in further detail in Example 1, below.

FIG. 2A-C illustrates demonstrating the added value of protein localization for predicting immunogenicity in an independent validation dataset:

FIG. 2A-B graphically illustrate area under the receiver operating characteristic curve (AUROC) (FIG. 2A) and area under the precision-recall curve (AUPRC) (FIG. 2B) for the unseen validation dataset with and without parent protein location features; and

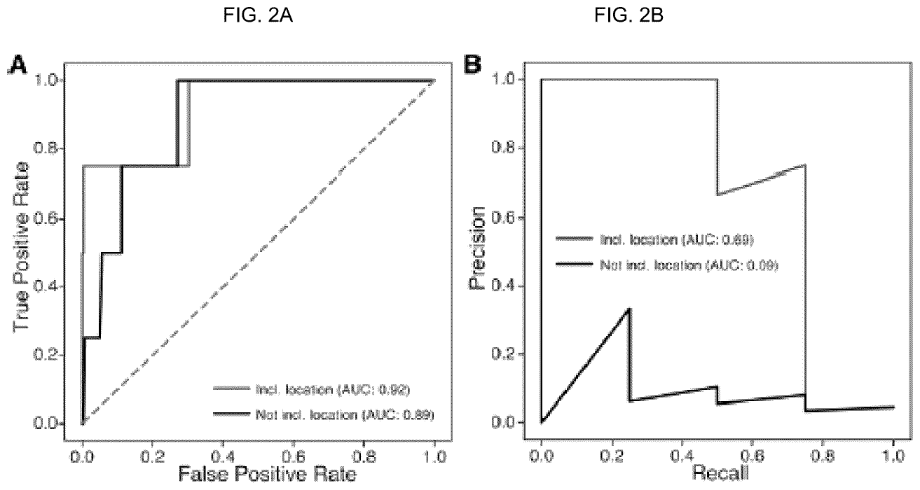

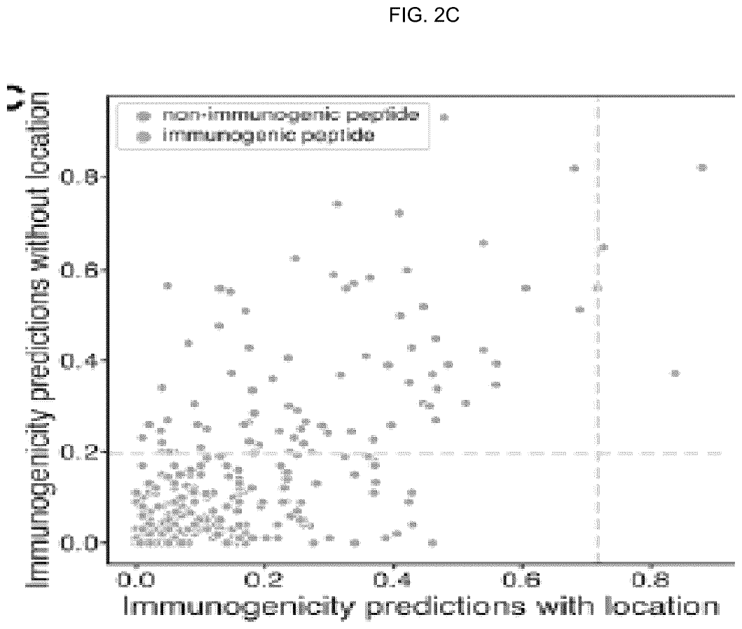

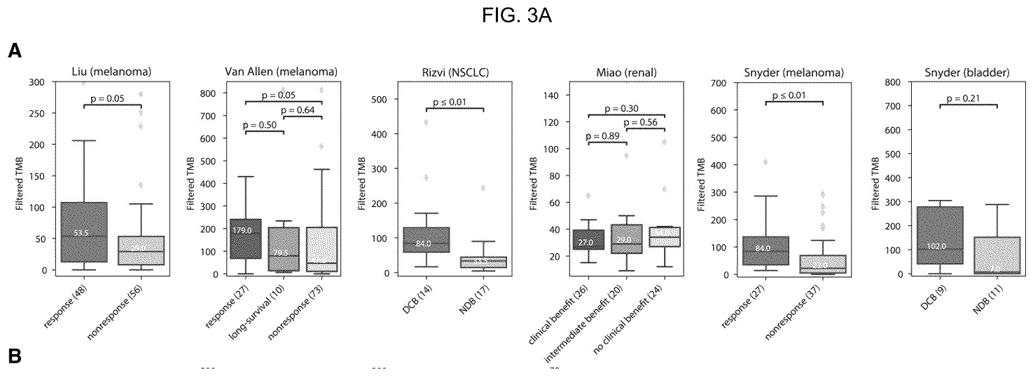

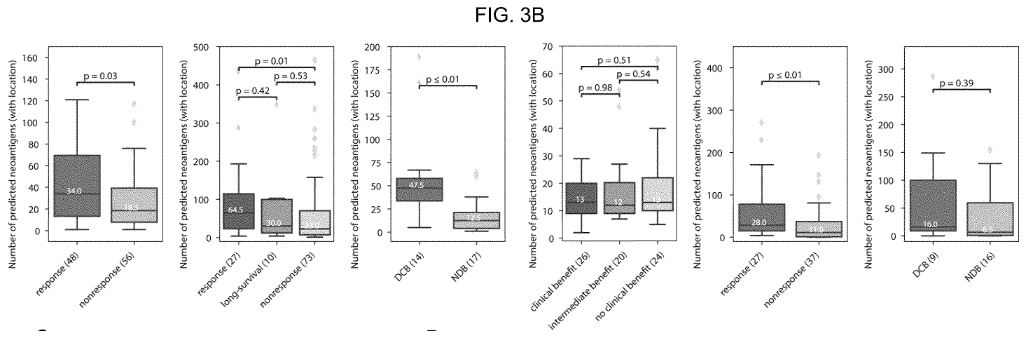

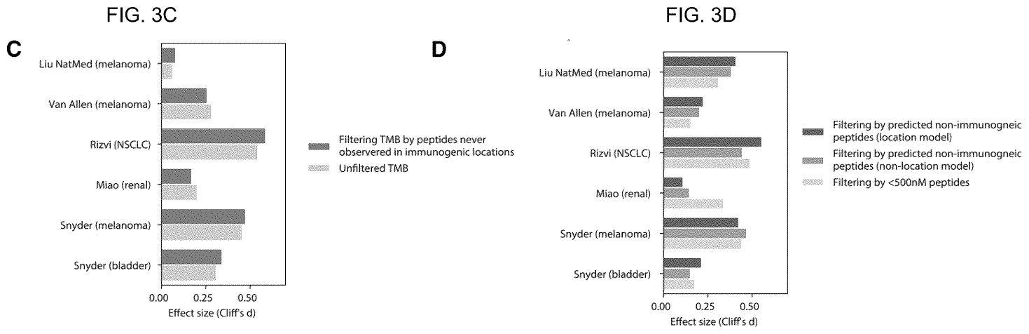

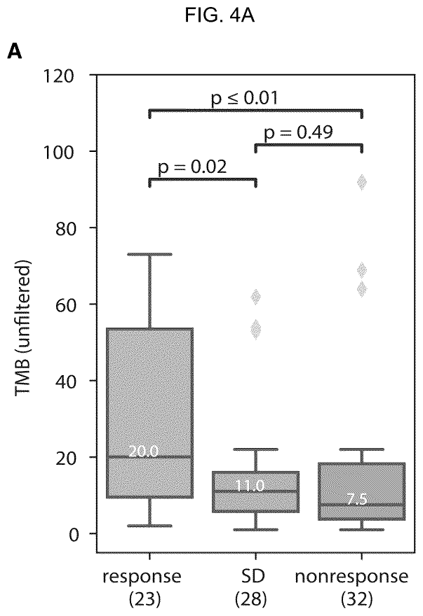

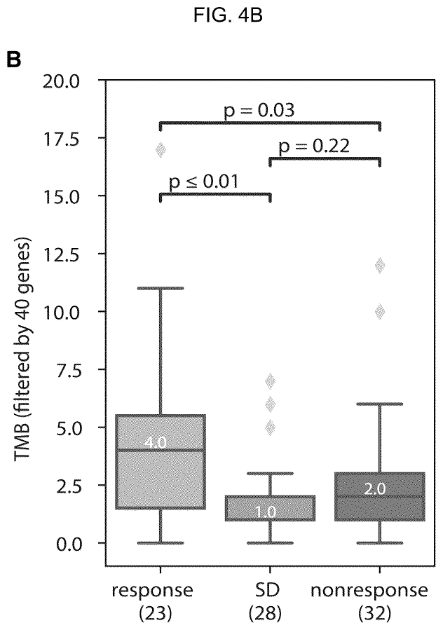

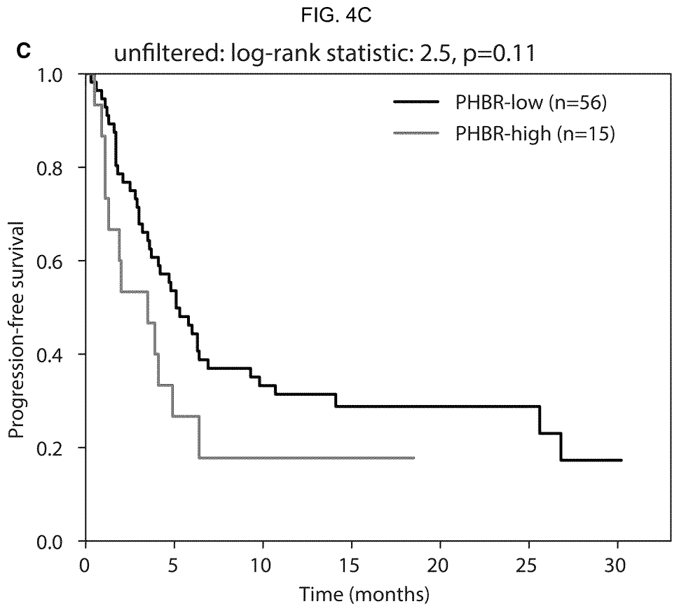

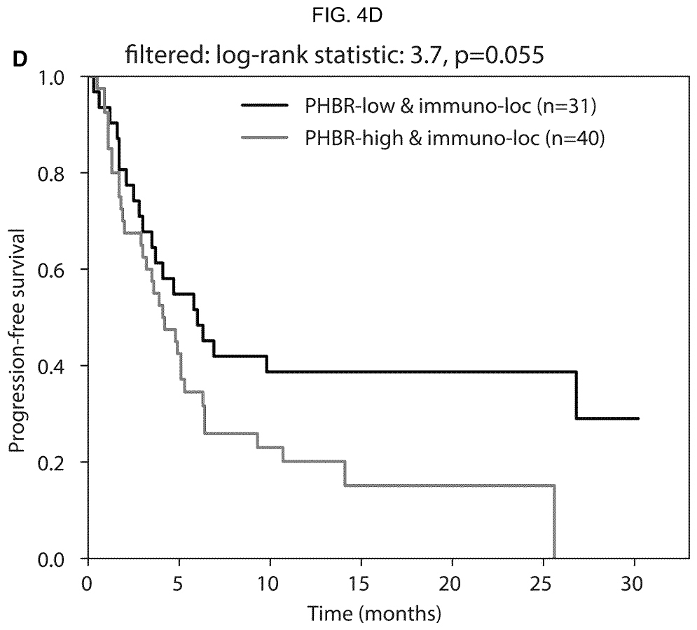

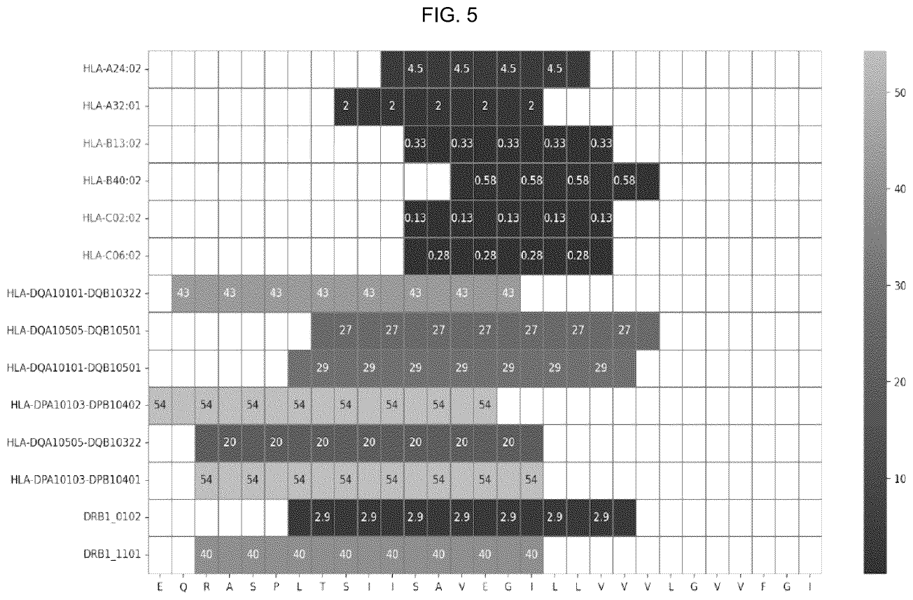

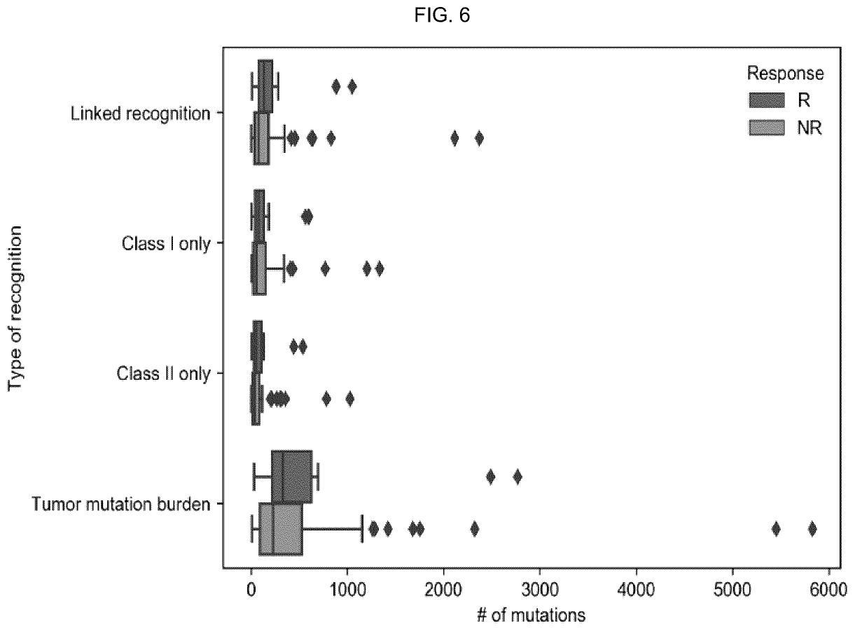

FIG. 2C graphically illustrates a scatterplot of the predicted probabilities for unseen test neopeptides to be immunogenic with and without location as a feature (immunogenicity predictions without location as a function of immunogenicity predictions with location); dashed lines indicate the Youden index (measures the effectiveness of a diagnostic marker and permits the selection of an optimal threshold value or cutoff point for the biomarker of interest) for each model, used for optimal threshold predictions; and false positives are reduced in the model with location,
as discussed in further detail in Example 1, below.

FIG. 3A-D graphically illustrate immune checkpoint inhibitor responders carry a higher burden of mutations in proteins from immunogenic locations:

FIG. 3A graphically illustrates predicted neoantigen burden versus response category in immunotherapy cohorts when retaining only mutations in proteins from subcellular locations previously observed to source immunogenic peptides;

FIG. 3B graphically illustrates predicted neoantigen burden versus response category in immunotherapy cohorts where neoantigen status is predicted using a model trained on three sources of immunogenic peptide and features including peptide-MHC affinity, stability, and location; and

FIG. 3C-D graphically illustrate barplots of effect sizes between responders and non-responders (FIG. 3C) where Tumor Mutational Burden (TMB) (represents the number of mutations per megabase (muts/Mb) harbored by tumor cells in a given neoplasm) is filtered to include only mutations from subcellular locations previously observed to source immunogenic peptides and (FIG. 3D) where neoantigen status is predicted using a model trained on three sources of immunogenic peptide and MHC affinity and stability, with and without location,
as discussed in further detail in Example 1, below.

FIG. 4A-D graphically illustrate focusing on immunogenic locations that improve immune checkpoint inhibitor response prediction in a gene panel profiled cohort:

FIG. 4A-B graphically illustrate tumor mutation burden focusing on (FIG. 4A) all genes in the gene panel and (FIG. 4B) the 40 genes whose proteins localize to previously observed immunogenic subcellular locations;

FIG. 4C-D graphically illustrate Kaplan-Meier curves (used to analyze time-to-event data, such as the time until death or the time until a specific event occurs) showing the effect of the best presented mutation on progression-free survival (FIG. 4C) using all genes in the panel and (FIG. 4D) using only the 40 genes of interest.

FIG. 5 illustrates a table showing a summary of the best binding peptides overlapping the BRAF V600E mutation for each MHC-I and MHC-II allele, as further discussed in Example 2, below.

FIG. 6 graphically illustrates the distribution of number of mutations generating LL neoantigens compared to mutations presented only by MHC-I, only MHC-II and all mutations compared between responders and non-responders to immune checkpoint inhibitor (ICI) therapy, as discussed in further detail in Example 3, below.

Like reference symbols in the various drawings indicate like elements.

### DETAILED DESCRIPTION

In alternative embodiments, provided are methods for identifying putative neoantigens based on two criteria: (i) potential for Linked recognition by CD4 and CD8 T cells; and (ii) intracellular Location of the source protein, and methods for using these neoantigens in disease therapies, for example, for identifying putative cancer neoantigens to be used in methods for treating cancer. Linked neopeptides with affinity for both MHC-I and MHC-II will produce neoantigens with superior immunogenicity.

The identification and selection of mutant peptides with immunogenic characteristics (neoantigens) remains complex, hindering the clinical success of neoantigen (personalized) vaccines. To overcome these barriers, we redesigned the process of selection of putative neoantigens to encompass additional new criteria.

Optimal T cell activation requires linked (or associative) recognition of antigen and cooperation between T cells, where both T cells respond to different antigenic peptides presented by the same antigen presenting cell. We posit that neoantigens need to obey to this fundamental rule of the adaptive T cell response. Considering all the factors that influence recognition of antigen and the complexity of selecting mutant peptides in a patient cancer genome, we propose two new criteria to identify and select neoantigens:
(i) potential for contextual-Linked recognition by CD4 and CD8 T cells; and
(ii) subcellular Location of the source protein, and
(iii) redundancy of presentation by MHC molecules.

We term this new class of neoantigens LL neoantigens.

As described in Example 1, below, we investigated whether constraints on peptide accessibility to the MHC due to protein subcellular location are associated with peptide immunogenicity potential. Analyzing over 380,000 peptides from studies of MHC presentation and peptide immunogenicity, we find clear spatial biases in both eluted and immunogenic peptides. We found that including parent protein location improves the prediction of peptide immunogenicity in multiple datasets. In human immunotherapy cohorts, the location was associated with a neoantigen vaccination response, and immune checkpoint blockade responders generally had a higher burden of neopeptides from accessible locations. We conclude that protein subcellular location adds important information for optimizing cancer immunotherapies.

We found that incorporating protein location into analysis of immunotherapy cohorts was helpful in several ways. We used location to revise the effective neoantigen burden in tumors and better stratify potential for immunotherapy response, although the best performance was observed in tumor types similar to the training data, namely melanoma, lung, and bladder, as well as datasets with higher overlap in the locations of the source proteins studied. Studying the effects of location in the context of tumor immunoediting is further made difficult by patterns of co-segregating mutation, and subclone-specific mechanisms of immune evasion can confound the association with neoantigen characteristics. More insight may be gained from future single-cell studies where it is possible to define the clonal architecture of tumors and determine which mutations coexist within the same clones. We found that location bias of mutated proteins correlated with immunoediting of specific tumor subclones in a murine model of melanoma. Location information was also beneficial in a cohort that was profiled with a gene panel, suggesting that this information could still be relevant for the more limited data commonly generated in clinical settings. Thus, we conclude that protein subcellular location contributes to shaping the tumor-immune interface and can potentially be leveraged to improve the effective application of immunotherapies.

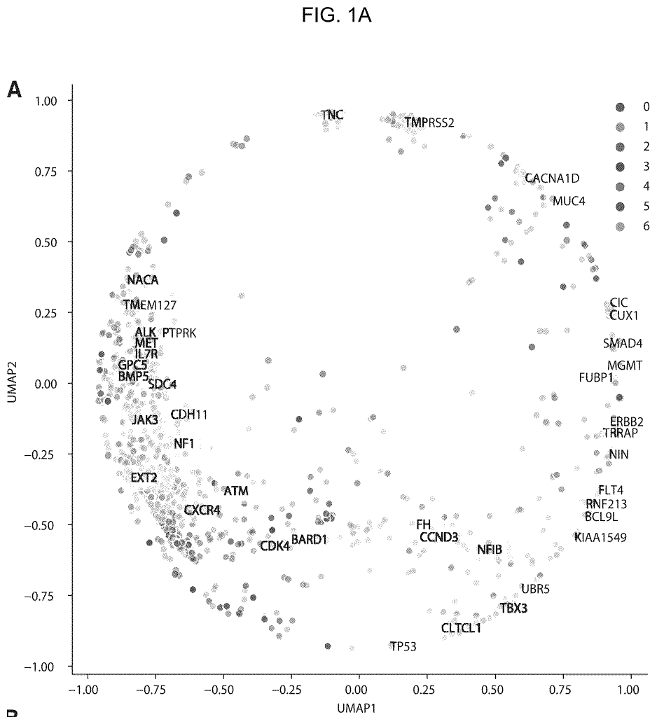

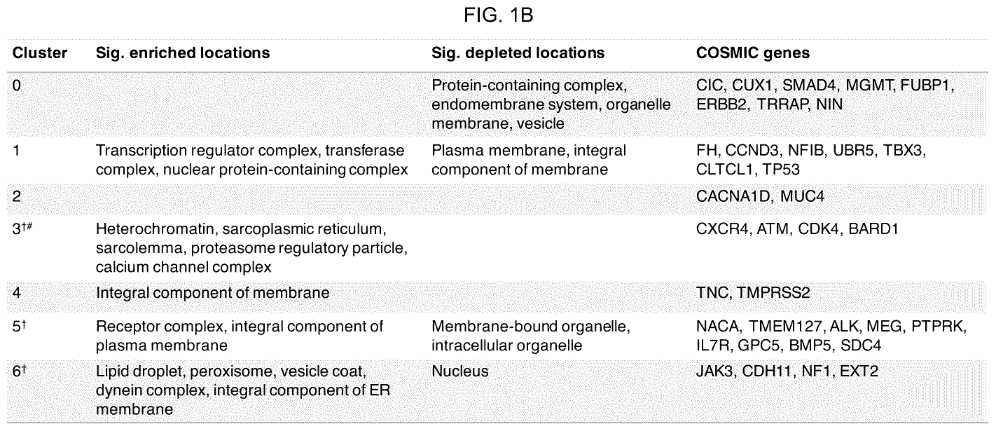

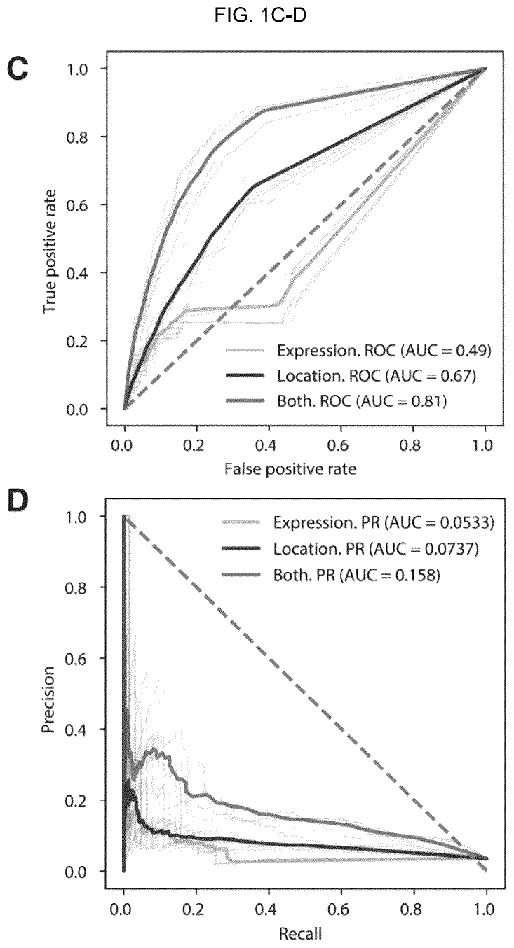

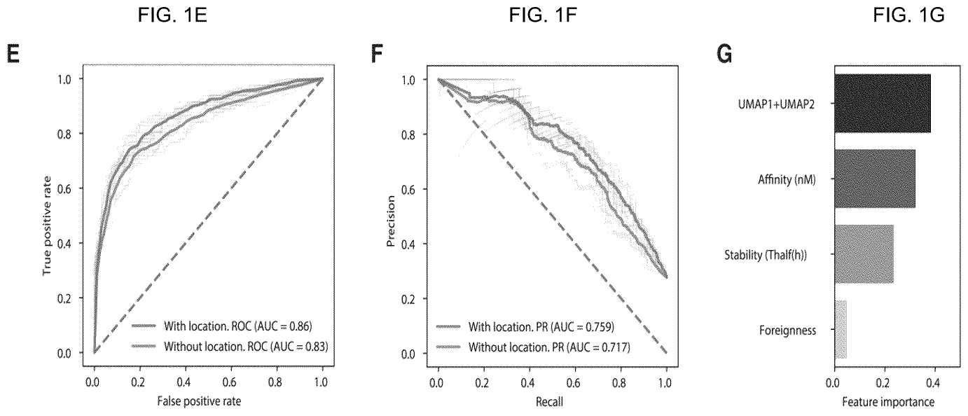

Provided herein are bioinformatic tools for the identification of neoantigens focused on Linked recognition and Location: LL neoantigens (FIG. 1, Example 2). Second, characterize the response of human T cells to LL neoantigens in the expectation that phenotype and strength are positively impacted. We believe that our new idea represents a sharp departure from current approaches, and integrates the mutational landscape of the cancer genome with the rules of activation of the T cell response and with protein traffic inside cancer cells as a new paradigm.

Provided herein are algorithms, and computer program products using these algorithms, to identify putative LL neoantigens in tumors that considers mutant protein expression, MHC-peptide affinity and stability, MHC integrity (i.e. accounts for somatic loss of HLA alleles), source protein localization and whether peptides have linked presentation (i.e., simultaneous neopeptide presentation by MHC-I and MHC-II). Source protein subcellular location is a novel determinant of immunogenicity. We compare linked neopeptides to those meeting the same criteria but presented exclusively by MHC-I and MHC-II. We focus on linked neopeptides associated with driver mutations and known cancer-related genes, and assess their relationship to immune infiltration and clinical outcome (FIG. 2, Example 2).

Provided herein are algorithms, and computer program products using these algorithms, for the prioritization of LL-neoantigens, determine whether T cell responding to LL neoantigens are phenotypically and/or functionally different from those generated against single MHC-I and MHC-II peptides of the respective proband LL neoantigen, and provide a molecular and transcriptional signature to optimize the induction of neoantigen-specific T cells in humans.

Provided herein are methods for treating infections and diseases such as cancer that can be treated by generating an immune response against an antigen using the optimization methods as provided herein for the induction of neoantigen-specific T cells in humans. LL neoantigens identified by methods and algorithms, and computer program products as provided herein can be used to induce T cells with superior characteristics, and thus develop superior immunotherapies. LL neoantigens identified by methods and algorithms, and computer program products as provided herein can be used to make improved LL-neoantigen-based vaccines.

In alternative embodiments, provided are methods for identifying and selecting an antigen that will be efficacious as an immune-stimulating antigen in vivo, wherein optionally the antigen that will be efficacious as an immune-stimulating antigen in vivo as (or in) a vaccine; and in alternative embodiments, the neoantigen is derived from, and/or the vaccine is effective against: a viral infection such as a coronavirus infection (such as COVID-19, or any of its variants, such as delta or omicron variants) or a microbial infection including a protozoan, helminthiasis, insect and/or parasitic infection such as: malaria that can be caused by a parasite of the genus Plasmodium (such as P. vivax, P. falciparum, P. malariae, P. ovale, or P. knowlesi); dengue fever; filariasis, leprosy or streptocerciasis that can be caused by a parasite of the superfamily Filarioidea (such as Brugia malayi, Brugia timori, Wuchereria bancrofti, Loa loa, Mansonella streptocerca, Mansonella ozzardi, or Mansonella perstans); leprosy that can be caused by a parasite of the genus Mycobacterium (such as M. leprae or M. lepromatosis); river blindness or onchocerciasis that can be caused by parasitic worms such as parasites of the genus Onchocerca (such as O. volvulus); hookworm or roundworm infections that can be caused by parasites of the genus Ancylostoma (such as A. duodenale or A. ceylanicum) or Necator (such as N. americanus); trichuriasis or whipworm infection that can be caused by a parasite of the genus Trichuris (such as T. trichuria); roundworm or an Ascaris infection that can be caused by Ascaris lumbricoides; mite-carried infections such as scabies that can be caused by the parasite of the genus Sarcoptes (such as S. scabiei); infections such as typhus caused by lice or parasites of the order Phthiraptera (such as Pediculus humanus capitis); enterobiasis that can be caused by pinworm or parasites of the genus Enterobius (such as E. vermicularis); pulicosis or infections cause by fleas or insects of the order Siphonaptera or of the genus Pulex (such as P. irritans), and other infections and infestations.

In alternative embodiments, provided are methods for identifying and selecting an antigen that will be efficacious as an immune-stimulating antigen in vivo, wherein optionally the antigen that will be efficacious as an immune-stimulating antigen in vivo against (to target) a cancer or tumor antigen, and optionally the immune-stimulating reaction comprises stimulating antigen-specific T cells, including T helper and/or T killer cells.

In alternative embodiments, methods as provided herein can identify neoantigens or antigens effective for generating an immune response (for example, a cell-based response, for example, a T cell response) to a cancer or tumor such as for example: acute lymphocytic cancer, acute myeloid leukemia, alveolar rhabdomyosarcoma, bladder cancer, bone cancer, brain cancer, breast cancer, cancer of the anus, anal canal, rectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal cancer, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, colorectal cancer, renal cancer, skin cancer, small intestine cancer, soft tissue cancer, solid tumors, stomach cancer, testicular cancer, thyroid cancer, ureter cancer, and/or urinary bladder cancer,

In alternative embodiments, antigens identified and selected using a method as provided herein are used to design and/or manufacture a chimeric antigen receptor (CAR) on a T cell, for example, a CAR-T cell, to generate a therapy that can be very effective in treating a cancer and another disease by the CAR-T′s ability to target a specific antigen, for example, a cancer antigen. Any method of making and using a CAR-T cell known in the art can be used with a method as provided herein to design a therapy for treating cancer, for example, as described in U.S. Pat. Nos. 9,328,156; 11,673,935; 11,377,637; or U.S. patent application publication no. 20230139800.

In alternative embodiment, provided are methods for making a vaccine comprising a neoantigen comprising: (a) identifying and selecting at least one neoantigen, or antigen identified as efficacious as an immune-stimulating antigen in vivo as (or in) a vaccine, using a method as provided herein; and, (b) formulating the at least one neoantigen or antigen with a vaccine formulation, optionally the vaccine formulation comprises a sterile saline.

In alternative embodiments, a nucleic acid encoding a peptide or polypeptide identified as an immune-stimulating antigen identified using a method as provided herein is used in a nucleic acid-based vaccine, optionally an RNA vaccine or a DNA vaccine.

In alternative embodiments, a vaccine designed by a method as provided herein can be administered orally or by inhalation, or can be administered by inclusion in a liquid (optionally to be administered as a drink or in drops such as nasal drops or in a mist), a tablet, a capsule, a gel, a geltab, a powder, a lozenge, an aerosol, spray, or mist formulation that is inhaled or administered nasally or orally (optionally, by a puffer or a nebulizer), or is formulated in a liquid (optionally the liquid is a sterile saline) solution which is ingested or gargled by the individual in need thereof.

Any method of making and using a peptide-based, DNA-based or RNA-based vaccine known in the art can be used with a method as provided herein, for example, as described in U.S. Pat. Nos. 5,837,249; 9,254,319; 9,353,159; or U.S. patent application publication nos. 20230346914; 20220323572; 20210162037.

### Products of Manufacture and Kits

Provided are products of manufacture and kits for practicing methods as provided herein; and optionally, products of manufacture and kits can further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: Subcellular Location of Source Proteins Improves Prediction of Neoantigens for Immunotherapy

This example demonstrates that identifying protein subcellular location adds important information for optimizing cancer immunotherapies.

### Methods:

We describe a method to represent proteins according to the locations inside the cell where they are found. We start with a list of cellular locations such as the cellular component annotation (CC) available through the Gene Ontology (GO) or similar or equivalent. We represent these locations as n-dimensional vectors using embedding techniques. The vectors associated with the CC terms in which a protein is a member are then summed into a single vector that describes the localization pattern for that specific protein. This can be weighted according to independent measures of abundance within each location when available. In order to obtain a quantitative parameter that can be used to encode a protein's unique localization pattern we perform dimensionality reduction over the protein vectors. We then use x and y coordinates in the reduced dimensionality space to represent the localization pattern of a protein.

We establish that this measure of localization pattern is predictive of whether a peptide from a given protein will be bound to MHC molecules on the cell surface (FIG. 1). We further show that neoepitopes derived from proteins that have a similar localization to proteins previously associated with epitope driven immune responses tend to be more predictive of immune outcomes (FIG. 2). We further show that this information is associated with response to neoantigen vaccine (Vaccine Evaluation). We further show that this information is useful for filtering mutations to remove those that will not be presented by the MHC. We further show that this filtering is predictive of outcome both for exome sequencing data and for panel sequencing data.

### Vaccine Evaluation:

We investigated the association of location with immune response in a neoantigen vaccine study (Sahin et al, 2017) and found that parent proteins of neopeptides able to induce a post-vaccination response ( 75/125 tested; 120 distinct parent proteins) were enriched for locations previously observed to contain immunogenic peptides from 3 datasets describing immunogenic neoantigen (Fisher's exact 3.49, P=0.029).

FIG. 1: illustrates an overview of location features from eluted peptides and T-cell assayed neoepitopes from IEDB:
A. Scatterplot of clustered UMAP location features (see Materials and Methods) for source proteins of the Immune Epitope DataBase (IEDB) T-cell assayed neoepitopes with COSMIC cancer genes highlighted.
B. Table annotating location clusters from the scatterplot with highlighted COSMIC (Catalogue Of Somatic Mutations In Cancer) (Wellcome Sanger Institute, Hinxton, Cambridgeshire, UK) cancer genes. †PANTHER GO SLIM CC™ (Protein ANalysis THrough Evolutionary Relationships Classification System) used here for simplified terms. #Only the top five are shown.
C, D. Area under the receiver operating characteristic curve (AUROC) and area under the precision-recall curve (AUPRC) for 10-fold cross-validation using a Random Forest model to predict protein elution in 721.221 B cells incorporating location, matched expression, or both (Materials and Methods). The faded lines indicate the respective area under the curve for each split.
E, F. AUROC and AUPRC for 10-fold cross-validation using a Random Forest model to predict immunogenicity in IEDB assayed neopeptides incorporating peptide affinity, stability, and foreignness (Materials and Methods) with and without parent protein location features.
G. Barplot of model feature importance.

### Example 2: Selecting Putative Neoantigens for Use in Immunotherapies

This example demonstrates that identifying peptides capable of linked MHC-I/MHC-II restricted recognition of mutated driver genes adds important information for optimizing cancer immunotherapies. This is a new approach to select clinically relevant neoantigens based on intracellular location of the source protein. We select putative neoantigens based on two new criteria: (i) potential for Linked recognition by CD4 and CD8 T cells; and (ii) intracellular Location of the source protein. Hence LL neoantigens.

### Methods:

A binding score is computed between each gene variant (allele) encoding an MHC molecule and all possible peptides overlapping a mutation capable of generating a neoantigen. For each allele, the best peptide is selected based on binding affinity between the peptide and the MHC produced by that allele. Additional information including proteosomal cleavage site, amino acid characteristics of the peptide, and location of the mutation at an anchor or non-anchor position can also be taken into account. The best peptide for each allele is then determined for each peptide, and assessed as to whether or not it is likely to be displayed by the MHC based on established binding affinity thresholds. Peptides that do not meet the criteria for binding to an allele are discarded. The redundancy score is the number of alleles that have a peptide that passes this criteria.

### Example:

Patient MHC alleles:
MHC-I
   A*24:02, A*32:01
   B*40:02, B*13:02
   C*02:02, C*06:02
MHC-II
   DPA1*01:03
   DPB1*04:01, DPB1*04:02
   DQA1*01:01, DQA1*05:05
   DQB1*03:22, DQB1*05:01
   DRB1*01:02, DRB1*11:01
Tumor Mutation: ERBB2: p.V659E
   wild-type peptide:
   EQRASPLTSIISAVVGILLVVVLGVVFGI (SEQ ID NO:1)
   mutated peptide:
   EQRASPLTSIISAVEGILLVVVLGVVFGI (SEQ ID NO:2)
Determine binding peptides for each MHC allele
   Each peptide has a binding score for each allele.
   First, the best binding peptide for each allele is determined.

In FIG. 5, MHC allele names are colored according to peptide binding:
Blue is weak binding (MHC-I<2; MHC-II<10)
Red is strong binding (MHC-I<0.5; MHC-II<2)

The amino acid sequence of the entire possible space of peptides is indicated on the x-axis. The best peptide is for each allele is indicated by the colored positions on the corresponding row. Peptides are colored according to binding affinity for each allele (left side color bar). Note: Low values indicate high affinity.

For this example the resulting redundancy scores are as follows:
MHC-I: 5,
MHC-II: 1,

### Example 3: Selecting Putative Neoantigens for Use in Immunotherapies

This example demonstrates that identifying peptides capable of linked MHC-I/MHC-II restricted recognition of mutated driver genes adds important information for optimizing cancer immunotherapies. This is a new approach to select clinically relevant neoantigens based on intracellular location of the source protein. We select putative neoantigens based on three new criteria: (i) potential for Linked recognition by CD4 and CD8 T cells; (ii) intracellular Location of the source protein; and (iii) redundancy of presentation by MHC. Hence LL neoantigens.

We evaluated the association of LL neoantigens with immune checkpoint inhibitor response in a set of 86 melanoma patients treated with anti-CLTA-4, see FIG. 6, which graphically illustrates the distribution of number of mutations generating LL neoantigens compared to mutations presented only by MHC-I, only MHC-II and all mutations compared between responders and non-responders to immune checkpoint inhibitor (ICI) therapy; the difference in burden between responders and non-responders was evaluated by Mann-Whitney U test. P-values: LL neoantinges p=0.066, MHC-I only p=0.201, MHC-II only p=0.075, overall TMB p=0.103. Thus, LL neoantigens showed the strongest association with ICI response in this dataset.

We also evaluated the association of linked recognition with T cell activation using a publicly available dataset with CD8 and CD4 T cell response information for specific peptide-MHC combinations.
1) Comparing CD8 T cell reactivity for peptides presented by MHC-I only versus by MHC-I and MHC-II simultaneously:

MHC-II presented peptides (PHBR-II < 10, num = 3721)
Contingency table

CD8 activity

| | | True | False |
|---|---|---|---|
| MHC-I NeoAg | True | 32 | 2656 |
| (PHBR-I score <2) | False | 1 | 1032 |

Chi-square test: p-value = 0.00278
odds ratio = 12.43

Not MHC-II presented peptides (PHBR-II >= 10, num = 3885)
Contingency table

CD8 activity

| | | True | False |
|---|---|---|---|
| MHC-I NeoAg | True | 20 | 2245 |
| (PHBR-II score <2) | False | 5 | 1615 |

Chi-square test: p-value = 0.0451
odds ratio = 2.88

2) Comparing CD4 T cell reactivity for peptides presented
by MHC-II only versus by MHC-I and MHC-II simultaneously:
MHC-I presented peptides (PHBR-I < 2, num = 4953)
Contingency table

CD4 activity

| | | True | False |
|---|---|---|---|
| MHC-II NeoAg | True | 41 | 2647 |
| (PHBR-II score <10) | False | 9 | 2256 |

Chi-square test: p-value = 0.000137
odds ratio = 3.88

Not MHC-I presented peptides (PHBR-I >= 2, num = 2653)
Contingency table

CD4 activity

| | | True | False |
|---|---|---|---|
| MHC-II NeoAg | True | 17 | 1016 |
| (PHBR-II score <10) | False | 4 | 1616 |

Chi-square test: p-value = 0.000184
odds ratio = 6.76

We observed a significantly higher odds of CD8 T cell activation when both MHC-I and MHC-II present a neoantigen (OR 12.43 vs OR 2.88 for MHC-I alone).

A number of embodiments of the invention have been described. Nevertheless, it can be understood that various modifications may be made without departing from the spirit and scope of the invention. Accordingly, other embodiments are within the scope of the following claims.

What is claimed is:

1. A method for identifying and selecting an antigen that will be efficacious as an immune-stimulating antigen in vivo,
wherein optionally the antigen that will be efficacious as an immune-stimulating antigen in vivo as (or in) a vaccine,
wherein optionally the antigen that will be efficacious as a T cell immune activating antigen,
the method comprising:
(i) providing a plurality of potential neoantigens, wherein optionally the plurality of potential neoantigens comprise a plurality of peptides, wherein optionally the peptides are between about 5 to 50 amino acids in length, or between about 2 to 25 amino acid in length;
(ii) identifying if there is a contextual-linked recognition of a candidate neoantigen by a CD4 and/or a CD8 T cell;
(iii) identifying if a source protein of the candidate neoantigen, or the neoantigen, is present in a subcellular location source known to contain immunogenic peptides, and
the neoantigen is enriched in at least one intracellular compartment or the neoantigen is depleted in at least one intracellular compartment,
and optionally the neoantigen is enriched when at least about 10%, 15%, 20% or 25% more amount of neoantigen is seen as compared to other neoantigens in the same cell compartment in the cell;
and optionally the neoantigen is depleted is when at least about 10%, 15%, 20% or 25% less amount of neoantigen is seen as compared to other neoantigens in the same cell compartment in the cell,
and optionally the cell compartment where the neoantigen is enriched is: cytoplasm, a NELF complex, nucleoplasm, a centrosome, a chromosome, a nucleolus, a nucleus or a combination thereof,
and optionally the cell compartment where the neoantigen is depleted is: Mitochondrial matrix and/or mitochondrion;
(iv) identifying if there is a redundancy of presentation of (or specific binding to) the neoantigen by at least two different major histocompatibility complex (MHC) proteins,
wherein optionally the neoantigen specifically binds a class 1 MHC polypeptide on a CD8+ cytotoxic T cell, and the neoantigen is a peptide 8 to 11 amino acids in length,
wherein optionally the neoantigen specifically binds a class II MHC polypeptide on a CD4+ helper T cell, and the neoantigen is a peptide 13 to 25 amino acids in length; and
(v) if a potential neoantigen meets the criteria of (ii), (iii) and (iv), the potential neoantigen is identified as an antigen that will be efficacious as an immune-stimulating antigen in vivo,
wherein optionally the immune-stimulation comprises generating a T cell based immune response, and optionally the T cell based immune response comprises a CD4+ helper T cell or a CD8+ cytotoxic T cell stimulation directed to the neoantigen.

2. The method of claim 1, wherein the neoantigen is a tumor or a cancer neoantigen.

3. The method of claim 1, wherein the neoantigen is derived from an infectious agent.

4. The method of claim 1, wherein the infectious agent is a virus, a bacteria, a protozoan or a fungus.

5. The method of claim 1, wherein the neoantigen is presented by (or is specifically bound by) a Class I MHC protein and a Class II MHC protein.

6. The method of claim 5, wherein the major histocompatibility complex (MHC) protein is a Class I MHC protein or a Class II MHC protein.

7. The method of claim 1, wherein the antigen and neoantigen is a tumor or cancer antigen and neoantigen.

8. The method of claim 5, wherein the tumor or cancer is a melanoma, lung or bladder tumor or cancer.

9. A method for designing a vaccine against a neoantigen comprising identifying and selecting a neoantigen using the method of claim 1.

10. A method for treating a tumor or a cancer comprising:
(a) exposing a T cell in vivo or ex vivo to a cancer or tumor neoantigen, wherein the cancer neoantigen is identified and selected using the method of claim 1;
wherein optionally the T cell is an autologous T cell, or the T cell is a CD4 or CD8 T cell; and
(b) (i) if the T cell is exposed ex vivo to the cancer or tumor neoantigen, administering the cancer or tumor neoantigen-exposed T cell to an individual in need thereof; or
(ii) if the T cell is exposed in vivo to the cancer or tumor neoantigen, a vaccine comprising the identified and selected cancer neoantigen is administered to the individual in need thereof.

11. A method for making a vaccine comprising a neoantigen comprising:
(a) identifying and selecting at least one neoantigen, or antigen identified as efficacious as an immune-stimulating antigen in vivo as (or in) a vaccine, using the method of claim 1; and,
(b) formulating the at least one neoantigen or antigen with a vaccine formulation, optionally the vaccine formulation comprises a sterile saline.

12. The method of claim 11, wherein the at least one neoantigen, or antigen identified as efficacious as an immune-stimulating antigen in vivo, comprises at least one peptide.

13. The method of claim 11, wherein the at least one neoantigen, or antigen identified as efficacious as an immune-stimulating antigen, or the at least one peptide, is formulated in a liposome, a polymeric nanoparticle or a virus-like particles (VLP).

14. The method of claim 11, wherein the vaccine further comprises an adjuvant or is administered to an individual in need thereof with an adjuvant, wherein optionally the adjuvant comprises an aluminum salt, MF59™ (or, squalene (4.3%) in citric acid buffer with stabilizing nonionic surfactants TWEEN 80™ (0.5%) and SPAN 85™ (0.5%)), and/or a CpG oligodeoxynucleotide.

15. The method of claim 11, wherein the at least one neoantigen, or antigen identified as efficacious as an immune-stimulating antigen in vivo, is loaded ex vivo onto a dendritic cell (DC), and the DC is thereupon administered to an individual in need thereof.

16. A method for treating a cancer or a tumor, or an infectious disease, comprising administering to an individual in need thereof a vaccine made by a method of claim 11, wherein optionally the vaccine is administered by a subcutaneous, intramuscular, or intranasal route.

17. A method for making a personalized adoptive cell therapy with an autologous T cell rendered neoantigen specific and expanded ex vivo comprising:
(a) identifying and selecting at least one neoantigen, or antigen identified as efficacious as an immune-stimulating antigen using the method of claim 1;
(b) stimulating ex vivo an autologous T cell from an individual in need thereof with at least one neoantigen, or antigen identified as efficacious as an immune-stimulating antigen; and
(c) administering to an individual in need thereof the ex vivo-stimulated autologous T cell.

18. The method of claim 17, wherein the ex vivo neoantigen-stimulated autologous T cell is administered directly into or approximate to (optionally within between about 1 to 50 mm to) a tumor site, or infected site, of an individual in need thereof.

19. The method of claim 17, wherein the ex vivo-stimulated autologous T cell is administered to the individual in need thereof with an immune checkpoint inhibitor, and optionally the immune checkpoint inhibitor comprises an anti-PD-1 or anti-CTLA-4 antibody.
